(19) United States
(12) Patent Application Publication
(10) Pub. No.: US 2026/0179732 A1
(43) Pub. Date: Jun. 25, 2026

(54) CLASSIFIER GUIDANCE OF DIFFUSION MODELS FOR CONTROLLED MOLECULE GENERATION

(71) Applicant: NEC Laboratories America, Inc., Princeton, NJ (US)

(72) Inventors: Tianxiao Li, Plainsboro, NJ (US); Renqiang Min, Princeton, NJ (US)

(21) Appl. No.: 19/417,508

(22) Filed: Dec. 12, 2025

Related U.S. Application Data
(60) Provisional application No. 63/736,104, filed on Dec. 19, 2024.

(51) Int. Cl. G16C 20/50; G16C 20/70

(52) U.S. Cl. CPC G16C 20/50; G16C 20/70

(57) ABSTRACT

Methods and systems for molecule generation include filtering a set of molecular properties to remove properties with low variance. The filtered set of molecular properties is embedded to a vector in a disentangled semantic space generating a molecule using an encoder. A new molecule from the vector is generated using a diffusion model.

### RELATED APPLICATION INFORMATION

This application claims priority to U.S. Patent Application No. 63/736,104, filed on Dec. 19, 2024, incorporated herein by reference in its entirety.

### BACKGROUND

### Technical Field

The present invention relates to molecular design and, more particularly, to classifier guidance of diffusion models for controlled molecule generation.

### Description of the Related Art

Computational design of molecules has roles in drug design and protein engineering. The three-dimensional geometry of molecules has implications for their properties and functions, such as quantum chemical properties, molecular dynamics, and interactions with protein receptors. Generative modeling of three-dimensional molecules seeks to create new molecules that have particular properties.

### SUMMARY

A method for molecule generation includes filtering a set of molecular properties to remove properties with low variance. The filtered set of molecular properties is embedded to a vector in a disentangled semantic space generating a molecule using an encoder. A new molecule from the vector is generated using a diffusion model.

A system for molecule generation includes a hardware processor and a memory that stores a computer program. When executed by the hardware processor, the computer program causes the hardware processor to filter a set of molecular properties to remove properties with low variance, to embed the filtered set of molecular properties to a vector in a disentangled semantic space generating a molecule using an encoder, and to generate a new molecule from the vector using a diffusion model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of molecule generation using a filtered set of molecular properties, in accordance with an embodiment of the present invention;

FIG. 2 is a block diagram of controlled molecule generation using a filtered set of molecular properties, in accordance with an embodiment of the present invention;

FIG. 3 is a block/flow diagram of a method for generating molecules, in accordance with an embodiment of the present invention;

FIG. 4 is a block diagram showing the use of a diffusion model with a classifier for molecule generation, in accordance with an embodiment of the present invention;

FIG. 5 is a block diagram showing a healthcare facility that uses controlled molecule generation for treatment, in accordance with an embodiment of the present invention;

FIG. 6 is a block diagram of a computing device that can perform controlled molecule generation, in accordance with an embodiment of the present invention;

FIG. 7 is a diagram of an exemplary neural network architecture that can be used to implement part of a diffusion model, in accordance with an embodiment of the present invention; and

FIG. 8 is a diagram of an exemplary deep neural network architecture that can be used to implement part of a diffusion model, in accordance with an embodiment of the present invention.

### DETAILED DESCRIPTION OF PREFERRED EMBODIMENTS

Molecular generation may be performed using diffusion models, guided by a property profile. The profile may include a set of properties that comprehensively cover the molecular space and could effectively control the majority of the aspects of generated molecules, including but not limited to their compositions, three-dimensional shapes, and physiochemical properties.

In some embodiments, the property profile may include a set of properties derived from open source tools, covering a wide range of molecular features. A comprehensive property profile can be flexibly adapted to generating new molecules or to manipulating existing models, without a need to train new models. Property manipulation may modify desired properties while preserving other properties, which can be achieved by modifying the desired properties in an existing property profile and using the new profile as classifier guidance.

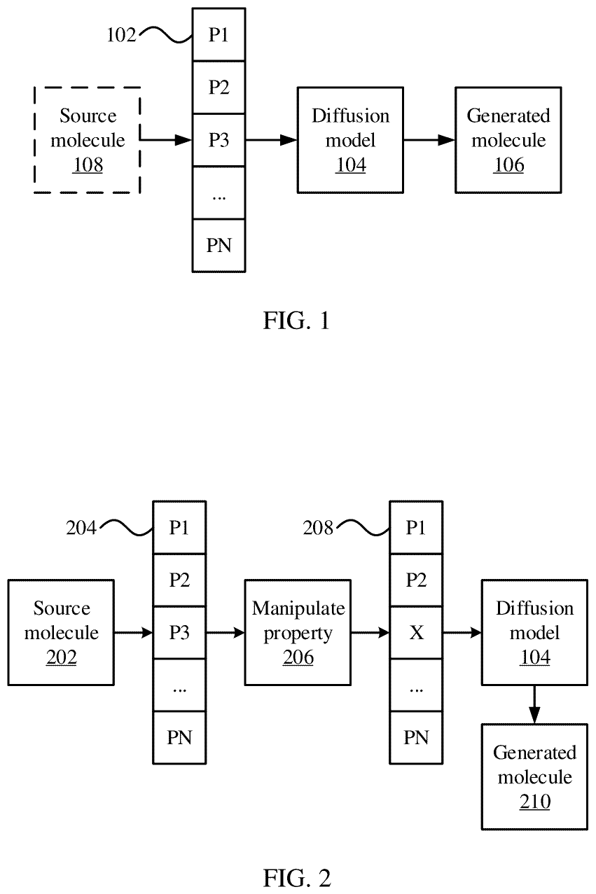

Referring now to FIG. 1, an example of molecule generation is shown. A property profile 102 includes a set of properties that relate to desired features (or forbidden features) that are to be included in the generated molecule 106. The property profile 102 is used as input to a diffusion model 104 which produces the generated molecule 106 to match the property profile 102.

The property profile 102 may be represented as a semantic embedding to control the direction of the generation towards specified molecular property profiles. In some embodiments the property profile 102 may be derived from a source molecule 108, for example by identifying the properties of the source molecule 108. The generated molecule 106 will thus be similar to, but not identical to, the source molecule 108 and will have similar properties. In other embodiments, the property profile 102 may be defined without a source molecule 108 as a starting point, instead being a set of desired properties without reference to a starting point.

Referring now to FIG. 2, an example of controlled property manipulation is shown. In this case, a source molecule 202 is used to generate an initial property profile 204. Block 206 manipulates one or more properties of the initial property profile 204 to produce a new property profile 208. The new property profile 208 is then used as input to the diffusion model 104 to generate a generated molecule 210 which is based on the source molecule 202 but which differs according to the manipulated property.

Semantics-guided controlled generation and property manipulation has control over a comprehensive set of molecule properties, rather than being conditioned on a single property. Disentanglement may be used to ensure that the properties can be selected independently of one another, ensuring that multiple objectives can be achieved without interference between them.

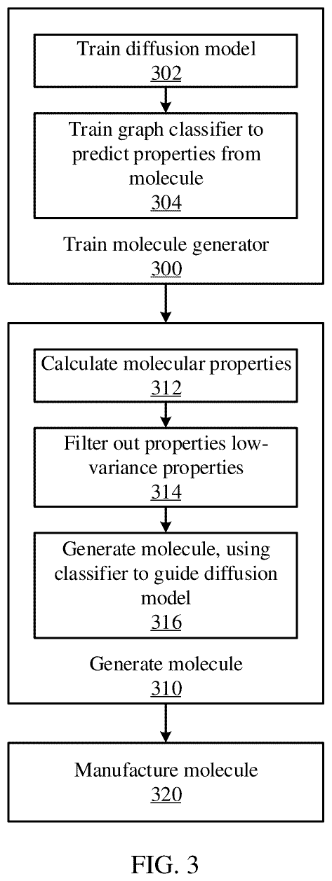

Referring now to FIG. 3, a method of training and using a diffusion model for molecule generation is shown. Block 300 trains a molecule generator that includes a diffusion model and a classifier. The diffusion model 104 may be trained in block 302 on a three-dimensional molecular conformational dataset, such as the GEOM dataset. Each molecule may be represented by its atom types, bond types, and three-dimensional coordinates. The data may be corrupted by time-dependent noise, so that the diffusion model 104 can be trained to predict the raw data from the corrupted data, conditioned on the time point and semantics embedding of the property profile. Block 304 trains an independent classifier based on equivariant graph neural networks (EGNNs) to predict the properties of an input three-dimensional molecule. To align the classifier with the diffusion model 104, the input may be corrupted with the same random noise as the diffusion model.

The training may use a set of drug-like molecules from a publicly available dataset. As noted above, a property dataset may be constructed for fine-tuning and training of the property classifiers, calculating the property of the validations et of the training dataset and holding out some data points for cross-validation. The size of the resulting dataset may be a fraction (e.g., 10%) of the training dataset. The test set may be used as test data for property manipulation tasks. In some cases, the molecules may be altered for computational simplicity, such as by removing hydrogens. Each three-dimensional molecule may be represented as a fully connected graph x=(r,h,E), where r is the coordinates of the atoms, h is the atom type, and E is an edge type. The coordinates r may be centered on the center of mass of the molecule, while h and E may be represented by one-hot encoding. Aromatic bonds may be considered a distinct type.

Block 310 then uses the trained system to generate new molecules. Block 312 creates a property profile, for example drawing properties from an initial property profile set. This property profile may then be filtered in block 314 to remove properties having properties with low variance, for example with variance of zero for continuous variables and with variance below a threshold (e.g., 0.1) for variables with discrete values. This produces a molecule's property profile with a large number of properties (e.g., 205 after an initial set of 222 is filtered). Block 316 then generates a molecule with the trained diffusion model 104, using the trained classifier to guide the diffusion. This process may begin with random noise, and the diffusion model iteratively modifies that initial noise to bring it into closer alignment with the property profile, with the classifier being used to determine what properties are captured by the present state of the molecule. This framework may also be used to manipulate properties, by obtaining the property profile from a known molecule and then modifying the values of particular dimensions as a target of the classifier guidance. Generated molecules may be evaluated according to a mean absolute error between their property values and target values.

Once the three-dimensional structure of the new molecule has been generated, the molecule may be generated in block 320 and may be used for any appropriate purpose, such as testing and administering the molecule for pharmaceutical purposes.

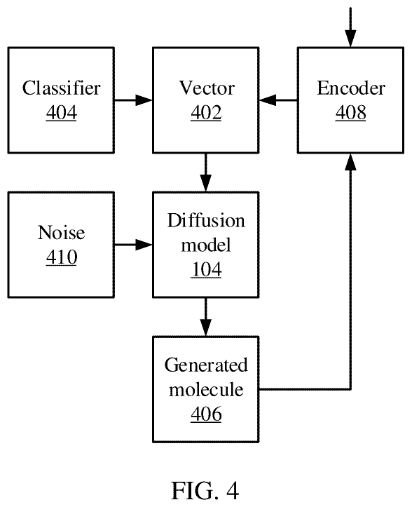

Referring now to FIG. 4, additional detail on the diffusion model 104 is shown. A vector 402 represents a set of molecule properties after they have been embedded in some latent space by an encoder 408. Initially the encoder 408 accepts the property profile and generates the vector 402. In subsequent iterations, the encoder 408 may process generated molecule 406 from a previous iteration to represent the properties of that most recent molecule.

The diffusion model 104 starts from noise 410, modifying the values of the initial noise to bring them closer to a form that represents the properties encoded in vector 402 to generate a molecule 406. This iterative process repeats until the generated molecule comes within some threshold similarity to the input properties. The diffusion model 104 may be implemented as a denoising diffusion implicit model or a denoising diffusion probabilistic model.

This process is an autoencoding framework that uses the semantic embedding to control the generation of the diffusion model 104. The semantic embedding of the vector 402 includes the complete molecular information and controls all aspects of the generated molecule 406. The disentanglement of the semantic embedding space dictated by the encoder 408 facilitates manipulation with multiple objectives, encouraging the diffusion model 104 to find directions that achieve all objectives with minimal conflicts.

The encoder 408 learns the higher-level semantics of an input 3D molecule or property profile, which is then provided to the diffusion model 104 as a condition. The diffusion model 104 functions as a decoder that translates the semantic embedding back to the three-dimensional molecular space. During training, the diffusion model 104 attempts to predict the clean input data from corrupted data and the semantic embedding. A Wasserstein loss may be used to minimize the distance between the distribution of the semantic latent space and a prior distribution p(z). This prior distribution p(z), may be an isotropic Gaussian distribution [symbol] (0,I). This regularization ensures maximal mutual information between the embedding and the input and also achieves disentanglement between the dimensions, leading to an objective function:

$$L = {L}_{D} ( θ ) + β {L}_{Wass}$$

where L_{D}(θ) is a diffusion loss, L_{Wass} is a Wasserstein loss, and β>0 is a regularization coefficient. The semantic encoder and the diffusion decoder are trained together on unlabeled three-molecules molecules.

For downstream tasks, the pretrained diffusion model 104 may be fine-tuned on datasets labeled with molecular properties. This may be accomplished using the classifier 404 on top of the semantics embedding to predict the target property, with the classifier 404, the encoder 408, and the diffusion model 104 being trained together with an additional classifier loss term L_{cls} is:

$$L = {L}_{D} ( θ ) + β {L}_{Wass} + {β}^{′} {L}_{cls}$$

where β′ is a weighting hyperparameter. The classifier 404 may take as input a molecular semantic embedding z. The classification loss is back-propagated to the semantic encoder 408 to improve the representation of z, which then is provided as an input to the diffusion model 104.

Fine-tuning may be performed on a subset of the full property profile, for example QED (quantitative estimate of drug-likeness), SAS (synthetic accessibility score), and Log P (logarithm of the partition coefficient between octanol and water), as their classification performance using pre-trained embedding may be poor relative to the others. These properties involve intricate interactions between the structural patterns of the molecules which are challenging to learn without supervision.

The diffusion model 104 may then be used to flexibly perform controlled generation with the semantic embedding vector 402. As the embedding contains the complete information about a molecule, the generated molecule 406 will possess the properties encoded by the embedding. It is also possible to alter certain properties of interest by directly manipulating the vector 402. In this way, the challenging problem of controlled generation with diffusion models is transformed into a latent space optimization.

To probe the information content of the semantic embedding space, linear regression models may be trained to predict the molecular properties from the embeddings. Despite being trained in an unsupervised manner, the embedding could be used to accurately predict many of the properties. For each property, only a limited, distinct set of dimensions have strong contributions. The patterns for QED, SAS and Log P become more distinguished after fine-tuning, while the other properties remain unaffected. Both the embedding dimensions and the properties show distinct clusters by their differential contributions. Meanwhile, the dimensions have minimal internal correlations. These results indicate the successful disentanglement between the dimensions enforced by the Wasserstein loss.

For most of the properties, the embedding space shows strong separation of the high- and low-value groups for various properties, including both 2D compositions and 3D shapes. This shows the semantics embedding space learns manifolds related to the molecular property. More complex properties like QED, SAS and Log P are harder to capture with simple unsupervised learning, as these properties are inferred from intricate combinations of multiple molecular features. Nevertheless, supervised fine-tuning on those properties makes the latent space achieve much better separation and prediction and while not impacting the other properties

The semantics embedding z shares maximal mutual information with the input x₀. The embedding has an impact on the generation process of the equivariant diffusion model, which can be used for the generation of 3D molecules with full molecular information provided.

The semantics embedding vectors 402 (z) of known source molecules 202 (x₀) are obtained from the encoder 408 to perform controlled diffusion generation using the diffusion model 104. To evaluate probabilistic models, a set of molecules may be generated for each embedding. An implicit model will generate near-perfect reconstructions, while a probabilistic model generates molecules that are sufficiently close to the source molecule in terms of composition and three-dimensional shape. The embedding z therefore dominates the denoising process, while the random noise

$${x}_{T}^{′}$$

accounts for minor variations.

When using the semantic embedding to modify some properties of interest, while maintaining the rest, the source embedding is represented as z and the target value for the properties is represented as

$${y}_{p}^{′} = ( {y}_{p 0}^{′} , … , {y}_{{p}_{m}}^{′} ) ,$$

where pᵢ is a property from the filtered set of properties [symbol] ₘ. The latent space is searched for a new embedding z′ that has

$${y}_{p}^{′}$$

while preserving the other properties.

In property manipulation tasks, a deterministic implicit model generation process may be used instead of a probabilistic model for better control, as the probabilistic model would introduce stochasticity during the denoising process. The diffusion model is deterministically reversed to map the source molecule x₀ to a noise point, performing denoising conditioned on the manipulated embedding z′. Some embodiments may perform a linear manipulation, where a linear regression model is trained to predict the property and then a constrained optimization problem is solved to find a closest z′ that contains the target property value. Some embodiments may perform classifier back-propagation, where the classifier is used to back-propagate the classification loss L_{cls} to z iteratively for the fine-tuning properties. The disentangled semantic space can successfully guide the diffusion model in multi-objective tasks. The three-dimensional shape of the source molecule is well-preserved in the generated molecule.

The encoder 408 takes an input molecule x₀=(r₀,h₀,E₀). The encoder 408 has an equivariant backbone that learns the conditional distribution of the semantics embedding z given the input:

$$q ( z \vert {x}_{0} ) = 𝒩 ( {μ}_{z} , {σ}_{z} ) {μ}_{z} , {σ}_{z} = {Encoder}_{γ} ( {x}_{0} ) .$$

where μ_{z} is a mean and σ_{z} is a standard deviation. The embedding z is then sampled from the distribution and provided to the diffusion model 104, which acts as a decoder to generate a reconstruction of the input. The embedding z is treated as a condition of the diffusion process. In practice, z is deterministically calculated from the input molecule without sampling.

In addition, the encoder can be co-trained with an auxiliary classifier ψ_{cls} is that predicts molecular properties y of interest from z, encouraging z to also carry information about y. The mean squared error (MSE) may be used as the auxiliary classification loss L_{cls}.

The diffusion model 104 may use a denoising diffusion probabilistic model with the semantics embedding as the condition. The conditional data distribution p_{θ}(x₀|z) is approximated through a series of latent variables x₁, . . . , x_{T} with the same dimension as x₀, named the reverse process, starting from a random noise point x_{T}:

$${p}_{θ} ( {x}_{0} \vert z ) = \int p ( {x}_{T} ) {\prod }_{t = 1}^{T} {p}_{θ} ( {x}_{t - 1} \vert {x}_{t} , z ) {dx}_{1 : T}$$

where T is a maximum number of denoising time steps in the diffusion model 104.

The posterior q(x_{1:T}|x₀,z), or the forward process gradually adds noise to x₀ until it eventually becomes a random noise x_{T}:

$$q ( {x}_{1 : T} \vert {x}_{0} , z ) = {\prod }_{t = 1}^{T} q ( {x}_{t} \vert {x}_{t - 1} , z )$$

The objective is to maximize the ELBO of log p(x₀)=log p(x₀|z)p(z). Under Gaussian assumptions, it is equivalent to minimizing the prediction loss of either the clean data x₀ or ϵₜ (the noise added to x₀ at time point t) from the corrupted data xₜ. The noise parametrization may be employed to achieve more stability in training. However, there is an advantage to using the clean data parametrization, as the model is aware of the overall graph structure throughout the training.

Based on these considerations, the diffusion model 104 is trained to predict the clean input x₀=(r₀, h₀, E₀) from corrupted data xₜ=(rₜ, hₜ, Eₜ), conditioned on the embedding z:

$${\hat{r}}_{0 , t} , {\hat{h}}_{0 , t} , {\hat{E}}_{0 , t} = {DM}_{θ} ( {r}_{t} , {h}_{t} , {E}_{t} , z , t )$$

Thus, the diffusion objective is defined as:

$${L}_{D} ( θ ) = {\sum }_{t = 1}^{T} {𝔼}_{( {r}_{0} , {h}_{0} , {E}_{0} ) , ( {\hat{r}}_{0 , t} , {\hat{h}}_{0 , t} , {\hat{E}}_{0 , t} )} [ {‖ {\hat{r}}_{0 , t} - {r}_{0} ‖}_{2}^{2} + {‖ {\hat{h}}_{0 , t} - {h}_{0} ‖}_{2}^{2} ] + {‖ {\hat{E}}_{0 , t} - {E}_{0} ‖}_{2}^{2} ]$$

In this case, z can be considered as controlling the direction of the denoising towards the target semantics. A regularization term is used to enforce the maximal mutual information (MI) between z and the input, which helps z to effectively guide and control the generation process.

To control the scale and shape of z, a Wasserstein loss is used on the marginal distribution q(z)=∫ᵣ_{₀}q_{γ}(z|x₀)q(x₀)dx₀. Specifically, a sample-based kernel maximum mean discrepancy (MMD) may be used on mini-batches of size n to make q(z) approach the shape of a Gaussian prior p(z)= [symbol] (0,I):

$${L}_{Wass} = MMD ( q ( z ) \vert \vert p ( z ) ) = \frac{1}{{n}^{2}} [ {\sum }_{i \neq j} k ( {z}_{i} , {z}_{j} ) + {\sum }_{i \neq j} k ( {z}_{i}^{′} , {z}_{j}^{′} ) - 2 \sum k ( {z}_{i} , {z}_{j}^{′} ) ]$$

where n is a number of samples in a minibatch, k is the kernel function, z₁'s are obtained from the data points in the minibatch and

$${z}_{1}^{′}$$

are randomly sampled from p(z)= [symbol] (0, I). This objective can be effectively calculated from the sample.

One concern with the diffusion autoencoder is that the diffusion model 104 may ignore the embedding z and solely rely on the noise x_{T} for the generation. The present objective also ensures maximal MI between z and the input x₀.

When β=1:

$$L = - {L}_{ELBO} - MI ( {x}_{0} , z )$$

$${L}_{ELBO} := - {L}_{D} ( {ϵ}_{θ} ) - {E}_{q ( {r}_{0} )} KL ( q ( z \vert {x}_{0} ) \vert \vert p ( z ) )$$

where KL is the Kullback-Leibler divergence, and L_{ELBO} is equivalent to the negative ELBO for log p_{θ}(r) under the model assumption. This means that by minimizing L, the ELBO and the MI may be jointly maximized. In practice, since KL(q(z)∥p(z)) is intractable, it may be approximated with an MMD. Both the KL divergence and the MMD are minimized when q(z)=p(z), thus justifying the approximation.

For semantics-guided stochastic generation, a probabilistic model may be used to progressively sample the clean data from random noise. For property manipulation tasks, the deterministic implicit sampling may be used. Starting from the random noise, a reconstruction of the clean data may be obtained by progressively removing the predicted noise deterministically:

$${v}_{t - 1} = \sqrt{{α}_{t - 1}} ( \frac{{v}_{t} - \sqrt{1 - {α}_{t}} {\hat{ϵ}}_{t}^{( v )}}{\sqrt{{α}_{t}}} ) + \sqrt{1 - {α}_{t - 1}} {\hat{ϵ}}_{t}^{( v )}$$

$${\hat{ϵ}}_{t}^{( v )} = \frac{1}{\sqrt{1 - {α}_{t}}} {v}_{t} - \frac{\sqrt{{α}_{t}}}{\sqrt{1 - {α}_{t}}} {\hat{v}}_{0 , t}$$

where v∈(r,h,E) and \hat{v}_{0,t} is the diffusion model prediction of the clean data from xₜ and z. This could also be re-written as:

$${v}_{t - 1} = \sqrt{{α}_{t - 1}} {\hat{v}}_{0 , t} - \frac{\sqrt{1 - {α}_{t - 1}}}{\sqrt{1 - {α}_{t}}} ( \sqrt{{α}_{t}} {\hat{v}}_{0 , t} - {v}_{t} )$$

where α is a time-dependent noise scale and v is the molecular data under denoising, which may include coordinate r, atom type h, and edge type E. The molecular data v plays the same role as the generic x in the formulation above.

When the time step is sufficiently small, the input data (r₀, h₀, E₀) can be mapped to the noise point (r_{T}, h_{T}, E_{T}) through an inverse of the denoising process:

$${v}_{t} = \sqrt{{α}_{t}} {\hat{v}}_{0 , t - 1} - \frac{\sqrt{1 - {α}_{t}}}{\sqrt{1 - {α}_{t - 1}}} ( \sqrt{{α}_{t - 1}} {\hat{v}}_{0 , t - 1} - {v}_{t - 1} )$$

Thus, when z remains unchanged in both processes, a perfect reconstruction has been achieved. When z is altered, the reverse process would be steered to a different direction than reconstruction.

The E(n)-equivariant graph neural network (EGNN) aims to incorporate geometric symmetry into molecular modeling. Given a fully connected graph [symbol] =(nᵢ,eᵢⱼ), i≠j where eᵢⱼ are edges and nᵢ are nodes with a coordinate rᵢ∈ [symbol] ³ and a feature hᵢ∈ [symbol] ^{d}, and a function (r′, h′)=f(r,h), the EGNN ensures the equivariance constraint. Namely, given an orthogonal matrix R∈ [symbol] ^{3×3} and a translation vector t∈ [symbol] ³:

$$R {x}^{′} + t , {h}^{′} = f ( R x + t , h ) .$$

In other words, h is invariant to transformations while x is receives an equivalent transformation. The EGNN may be used as the backbone of a DDPM for 3D molecules. With an invariant prior p(x_{T}), the equivariant process results in an invariant data distribution p_{θ}( [symbol] ), where the probability of a data point remains the same after [symbol] transformation. This greatly improves data efficiency and model generalizability.

Both the encoder 408 and the diffusion model 104 may use an EGNN backbone. The encoder 408 aggregates the invariant output of the EGNN as the semantic embedding:

$${z}^{( r )} , {z}^{( h )} , {z}^{( E )} = {EGNN}_{γ} ( {r}_{0} , {h}_{0} , {E}_{0} )$$

where z^{(r)} is equivariant and z^{(h)},z^{(E)} are invariant. The semantic embedding is calculated using a multi-layer perceptron (MLP) as:

$$z = M L P ( {z}^{( h )} , {z}^{( E )} )$$

Because all properties may only be related to the invariant features (atom types, edge types and pairwise distances), only the invariant part of the encoder output is kept as the semantic embedding. For generative tasks where 3D geometry and symmetry are involved, the equivariant output could also be used.

The diffusion model 104 uses the same EGNN architecture to predict the clean data x₀ from the noisy data xₜ. The semantic embedding z and the embedding of the time point t are concatenated to all node and edge features.

$${\hat{r}}_{0 , t} , {h}_{0 , t} , {\hat{E}}_{0 , t} = {EGNN}_{θ} ( {r}_{t} , {h}_{t} , {E}_{t} , z , t )$$

Since the DDIM-based diffusion inversion only works on continuous distributions, a continuous diffusion model may be used. The categorical values h and E are treated as multinomial Gaussian distributions of the respective dimensions.

The semantics embedding z of a known molecule x₀=(r₀, h₀, E₀) from the encoder may be combined with randomly sampled stochastic noise

$${x}_{T}^{′} = ( {r}_{T}^{′} , {h}_{T}^{′} , {E}_{T}^{′} )$$

to generate new molecules

$${x}_{0}^{′} = ( {r}_{0}^{′} , {h}_{0}^{′} , {E}_{0}^{′} ) .$$

Specifically, the embedding z may be determined for 100 source molecules, randomly selected from the training set, and 10 molecules may be generated for each z. The molecular properties of x₀ and

$${x}_{0}^{′}$$

are generated by calculating the mean absolute error (MAE) for each property against the respective source molecule. Properties with standard deviation <0.1 may be discarded and the remaining properties may be scaled by their standard deviation. As a random control, 10 random property profiles may be generated for each source from [symbol] (μₚ,σₚ) for each property p where μₚ,σₚ are the mean and standard deviation.

The average 2D and 3D Tanimoto similarity may be determined between the generated molecules and their sources. The random control is the average similarity of randomly sampled molecule pairs from the training set.

For manipulating semantics, given a known molecule, a linear framework may be used to manipulate the semantic embedding to gain certain properties. Linear regression is trained on the semantic embeddings to predict the property. Then, given a source embedding z, target value(s) y′, and the weight and bias of the linear regression (w,b) a new embedding z′ can be determined with the desired property via: z′←z+w⁺(y′−b−wz), where w⁺ is the pseudo-inverse of w. Then z′ minimizes ∥z′−z∥² subject to y′=wz′+b.

For the fine-tuning properties (e.g., QED, SAS, Log P), the auxiliary classifier ψₐᵤₓ can be used to directly manipulate the embedding through iterative back propagation. The embedding z′ is classified to produce y̅ and a mean squared error is determined between the output of that classification and y′. The embedding may be updated as $z′←z′+λ∇_{z′}L_{cls}, where λ is the learning rate and n is a number of iterations. For example, for QED the values of n=20 and λ=1.0 may be used, but λ=0.1 may be used for SAS and Log P.

To decode the manipulated embedding, x₀ may first be reverse mapped to a noisy data point xₜ with the source embedding z. The manipulated embedding z′ and xₜ may be provided to the diffusion decoder 104 to generate a manipulated molecule

$${x}_{0}^{′} = ( {r}_{0}^{′} , {h}_{0}^{′} , {E}_{0}^{′} ) .$$

In practice, the full set of inputs need not be reverse-mapped. Instead, up to x_{T/2} may be reverse mapped instead of up to x_{T}. Denoising may then be performed for T/2 steps without skipping, to give the best generation quality.

Given a target property p, the molecules which have a respective property value above or below the mean μₚ may be selected, and the target value may be set as y′=μₚ+2σₚ. The same applies in multi-objective tasks.

For a conditional diffusion model, for each property p, a diffusion model may be trained on the property dataset conditioned on the property value yₚ, with the forward and reverse processes are defined as:

$${p}_{θ} ( {x}_{0} \vert {y}_{p} ) = \int p ( {x}_{T} ) {\prod }_{t = 1}^{T} {p}_{θ} ( {x}_{t - 1} \vert {x}_{t} , {y}_{p} ) d {x}_{1 : T}$$

$$q ( {x}_{1 : T} \vert {x}_{0} , {y}_{p} ) = {\prod }_{t - 1}^{T} q ( {x}_{t} \vert {x}_{t - 1} , {y}_{p} )$$

In practice, the property value is combined with the embedding of the time point t and then concatenated to every node and edge feature.

For the property manipulation using the implicit model, x₀ is reverse mapped to a noisy data point xₜ with the source property yₚ. Reverse mapping may again be performed with T/2 steps. Then, xₜ is combined with the target value

$${y}_{p}^{′}$$

in the denoising process to obtain the manipulated molecule.

$${x}_{0}^{′} .$$

Classifier guidance may also be performed on the continuous version of the diffusion model. For property-controlled generation, train classifiers may be trained to predict all of the properties in the property profile. For property manipulation, separate classifiers may be trained for each target property. The classifiers have the same EGNN-based architecture as the semantic encoder 408, with a 2-layer MLP prediction head added on top of the model output.

Probabilistic models may be used for property-controlled generation and implicit models may be used with T/2 reverse and denoising steps for property manipulation. In either case, let xₜ be the prediction output of any denoising step, vₜ be one of (rₜ, hₜ, Eₜ), [symbol] ₘ be the set of manipulated properties,

$${y}_{p}^{′}$$

be the target value of property p and Ψₚ be the respective classifier, at every time step, xₜ is further updated with the classifier gradient:

$${v}_{t} ← {v}_{t} + {\sum }_{p ∈ {ℱ}_{m}} {λ}_{p} {∇}_{{v}_{1}} {L}_{cls} ( {Ψ}_{p} ( {x}_{t} ) , {y}_{p}^{′} )$$

where λ is the guidance strength and L_{cls} is the classifier loss, which is MSE. For property-controlled generation, min-max scaling may be performed on all properties with λ=0.1. For property manipulation, λ=0.01 for SAS and log P and λ=0.1 may be used for the other properties.

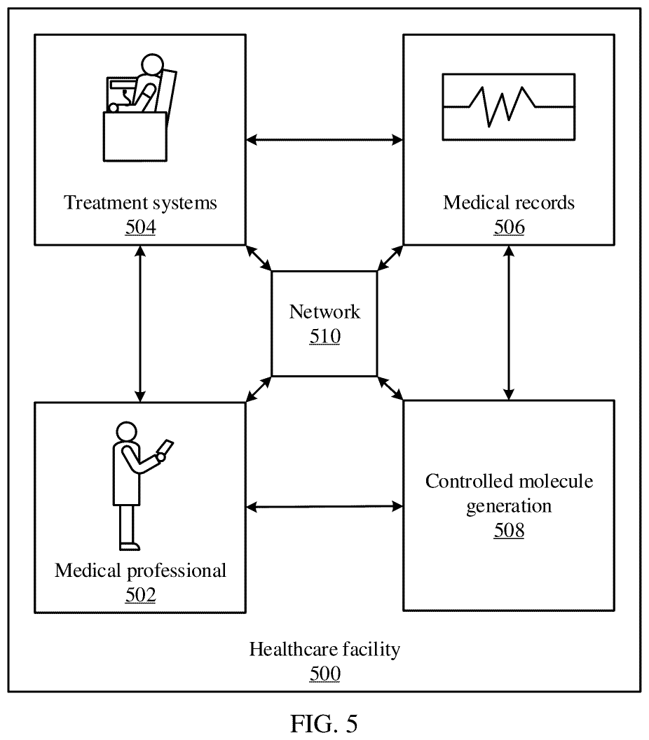

Referring now to FIG. 5, a controlled molecule generation is shown in the context of a healthcare facility 500. Controlled molecule generation 508 may be used to create a drug that is tailored to a particular disease or patient. For example, a specialized drug may be needed that has particular properties, and a new molecule may be generated from the list of properties or from a similar drug, altering specific properties of the existing drug to create a new drug.

The healthcare facility may include one or more medical professionals 502 who review information extracted from a patient's medical records 506 to determine their healthcare and treatment needs. These medical records 506 may include self-reported information from the patient, test results, and notes by healthcare personnel made to the patient's file. Treatment systems 504 may furthermore monitor patient status to generate medical records 506 and may be designed to automatically administer and adjust treatments as needed.

Using the generated molecule, the medical professionals 502 may then make medical decisions about patient healthcare suited to the patient's needs. For example, the medical professionals 502 may determine and administer a course of treatment based on the generated molecule.

The different elements of the healthcare facility 500 may communicate with one another via a network 510, for example using any appropriate wired or wireless communications protocol and medium. Thus controlled molecule generation 508 receives data from treatment systems 504, medical professionals 502, and from medical records 506 to create a molecule that has specified properties. The treatment systems 504 may be used to automatically administer or alter a treatment based on the generated molecule, such as by initiating or halting the administration of a medication.

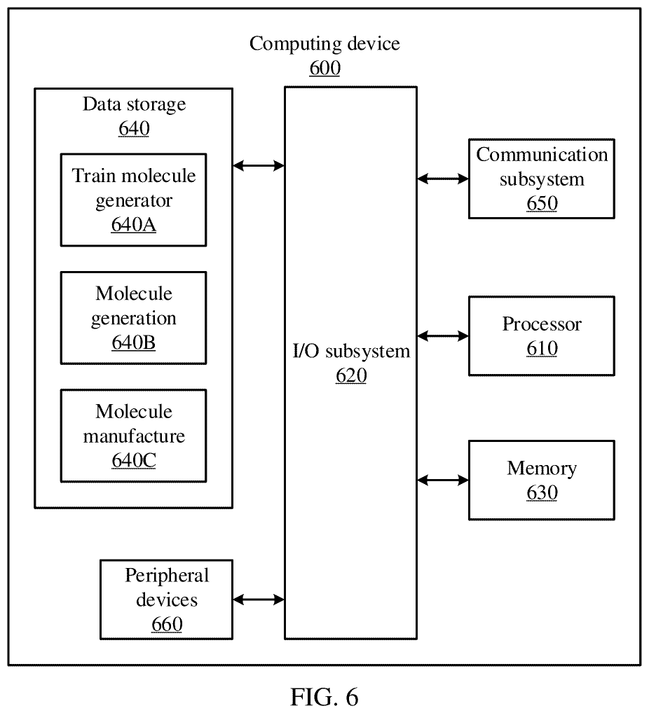

Referring now to FIG. 6, an exemplary computing device 600 is shown, in accordance with an embodiment of the present invention. The computing device 600 is configured to perform controlled molecule generation.

The computing device 600 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 600 may be embodied as one or more compute sleds, memory sleds, or other racks, sleds, computing chassis, or other components of a physically disaggregated computing device.

As shown in FIG. 6, the computing device 600 illustratively includes the processor 610, an input/output subsystem 620, a memory 630, a data storage device 640, and a communication subsystem 650, and/or other components and devices commonly found in a server or similar computing device. The computing device 600 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 630, or portions thereof, may be incorporated in the processor 610 in some embodiments.

The processor 610 may be embodied as any type of processor capable of performing the functions described herein. The processor 610 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 630 may be embodied as any type of volatile or non-volatile memory or data storage capable of performing the functions described herein. In operation, the memory 630 may store various data and software used during operation of the computing device 600, such as operating systems, applications, programs, libraries, and drivers. The memory 630 is communicatively coupled to the processor 610 via the I/O subsystem 620, which may be embodied as circuitry and/or components to facilitate input/output operations with the processor 610, the memory 630, and other components of the computing device 600. For example, the I/O subsystem 620 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 620 may form a portion of a system-on-a-chip (SOC) and be incorporated, along with the processor 610, the memory 630, and other components of the computing device 600, on a single integrated circuit chip.

The data storage device 640 may be embodied as any type of device or devices configured for short-term or long-term storage of data such as, for example, memory devices and circuits, memory cards, hard disk drives, solid state drives, or other data storage devices. The data storage device 640 can store program code 640A for training a molecule generator, 640B for molecule generation, and/or 640C for manufacturing a molecule. Any or all of these program code blocks may be included in a given computing system. The communication subsystem 650 of the computing device 600 may be embodied as any network interface controller or other communication circuit, device, or collection thereof, capable of enabling communications between the computing device 600 and other remote devices over a network. The communication subsystem 650 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 600 may also include one or more peripheral devices 660. The peripheral devices 660 may include any number of additional input/output devices, interface devices, and/or other peripheral devices. For example, in some embodiments, the peripheral devices 660 may include a display, touch screen, graphics circuitry, keyboard, mouse, speaker system, microphone, network interface, and/or other input/output devices, interface devices, and/or peripheral devices.

Of course, the computing device 600 may also include other elements (not shown), as readily contemplated by one of skill in the art, as well as omit certain elements. For example, various other sensors, input devices, and/or output devices can be included in computing device 600, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 600 are readily contemplated by one of ordinary skill in the art given the teachings of the present invention provided herein.

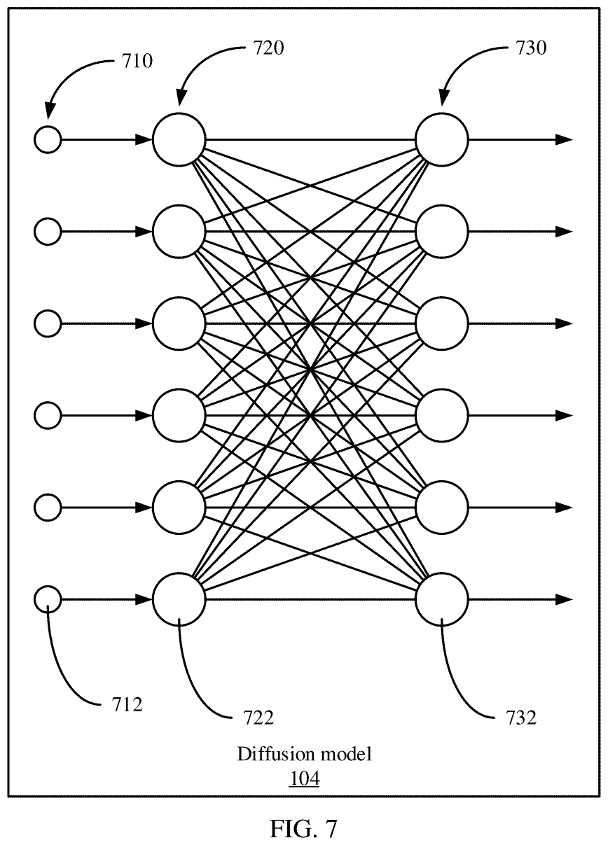

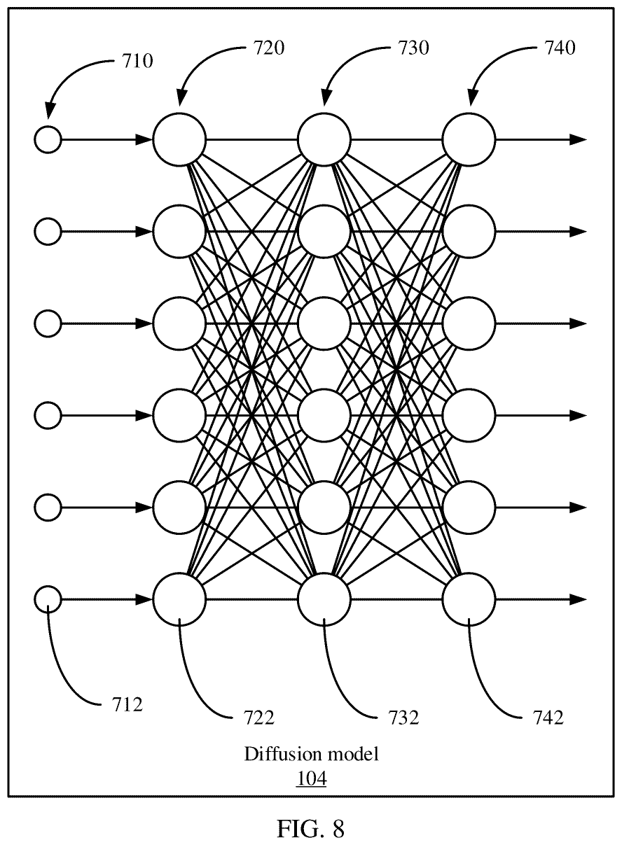

Referring now to FIGS. 7 and 8, exemplary neural network architectures are shown, which may be used to implement parts of the present machine learning models, such as the diffusion model 104. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 720 of source nodes 722, and a single computation layer 730 having one or more computation nodes 732 that also act as output nodes, where there is a single computation node 732 for each possible category into which the input example could be classified. An input layer 720 can have a number of source nodes 722 equal to the number of data values 712 in the input data 710. The data values 712 in the input data 710 can be represented as a column vector. Each computation node 732 in the computation layer 730 generates a linear combination of weighted values from the input data 710 fed into input nodes 720, and applies a non-linear activation function that is differentiable to the sum. The exemplary simple neural network can perform classification on linearly separable examples (e.g., patterns).

A deep neural network, such as a multilayer perceptron, can have an input layer 720 of source nodes 722, one or more computation layer(s) 730 having one or more computation nodes 732, and an output layer 740, where there is a single output node 742 for each possible category into which the input example could be classified. An input layer 720 can have a number of source nodes 722 equal to the number of data values 712 in the input data 710. The computation nodes 732 in the computation layer(s) 730 can also be referred to as hidden layers, because they are between the source nodes 722 and output node(s) 742 and are not directly observed. Each node 732, 742 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 732 in the one or more computation (hidden) layer(s) 730 perform a nonlinear transformation on the input data 712 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for molecule generation, comprising:
filtering a set of molecular properties to remove properties with low variance;
embedding the filtered set of molecular properties to a vector in a disentangled semantic space generating a molecule using an encoder; and
generating a new molecule from the vector using a diffusion model.

2. The method of claim 1, wherein generating the new molecule uses a classifier to guide the diffusion model by determining properties of a molecule output by the diffusion model.

3. The method of claim 2, wherein the classifier is fine-tuned on properties that include QED, SAS, and Log P.

4. The method of claim 2, wherein the classifier is implemented as an equivariant graph neural network and wherein the classifier and the diffusion model are trained together using training data that is corrupted by a same noise input.

5. The method of claim 2, wherein the classifier, the encoder, and the diffusion model are implemented using equivariant graph neural network layers.

6. The method of claim 2, wherein the classifier is trained using a same time-dependent noise signal as is used to train the diffusion model.

7. The method of claim 1, further comprising determining the filtered set of properties for a source molecule and altering at least one of the determined properties before embedding the filtered set of properties.

8. The method of claim 1, further comprising manufacturing the new molecule.

9. The method of claim 1, wherein filtering the set of molecular properties starts with 222 properties and produces a filtered set of 205 molecular properties that comprehensively cover a molecular space to control aspects of generated molecules.

10. The method of claim 1, wherein the new molecule is represented by atom types, bond types, and three dimensional coordinates.

11. A system for molecule generation, comprising:
a hardware processor; and
a memory that stores a computer program which, when executed by the hardware processor, causes the hardware processor to:
filter a set of molecular properties to remove properties with low variance;
embed the filtered set of molecular properties to a vector in a disentangled semantic space generating a molecule using an encoder; and
generate a new molecule from the vector using a diffusion model.

12. The system of claim 11, wherein generating the new molecule uses a classifier to guide the diffusion model by determining properties of a molecule output by the diffusion model.

13. The system of claim 12, wherein the classifier is fine-tuned on properties that include QED, SAS, and Log P.

14. The system of claim 12, wherein the classifier is implemented as an equivariant graph neural network and wherein the classifier and the diffusion model are trained together using training data that is corrupted by a same noise input.

15. The system of claim 12, wherein the classifier, the encoder, and the diffusion model are implemented using equivariant graph neural network layers.

16. The system of claim 12, wherein the classifier is trained using a same time-dependent noise signal as is used to train the diffusion model.

17. The system of claim 11, further comprising determining the filtered set of properties for a source molecule and altering at least one of the determined properties before embedding the filtered set of properties.

18. The system of claim 11, further comprising manufacturing the new molecule.

19. The system of claim 11, wherein filtering the set of molecular properties starts with 222 properties and produces a filtered set of 205 molecular properties that comprehensively cover a molecular space to control aspects of generated molecules.

20. The system of claim 11, wherein the new molecule is represented by atom types, bond types, and three dimensional coordinates.
